SEQUENCE LISTING (24 sequences)

SEQ ID NO: 1 - DNA, 393 bp, Mus musculus
atgagagtgctgattcttttgtggctgttcacagcctttcctggtatcctgtctgatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagctacagtggtagcactaactacaacccatctctcaaaagtcgaatctctatcactcgagacacatccaagaaccagttcttcctgcagttgaattctgtgactactgaggacacagccacatattactgtacaagagatgggggttactggggtcaaggaacctcagtcaccgtctcctca

SEQ ID NO: 2 - protein, 131 aa, Mus musculus
MRVLILLWLFTAFPGILSDVQLQESGPGLVKPSQSLSLTCTVTGYSITSDYAWNWIRQFPGNKLEWMGYISYSGSTNYNPSLKSRISITRDTSKNQFFLQLNSVTTEDTATYYCTRDGGYWGQGTSVTVSS

SEQ ID NO: 3 - DNA, 399 bp, Mus musculus
atggattcacaggcccaggttcttatgttactgctgctatgggtatctggtacctgtggggacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctctcacgttcggtgctgggaccaagctggagctgaaa

SEQ ID NO: 4 - protein, 133 aa, Mus musculus
MDSQAQVLMLLLLWVSGTCGDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLELK

SEQ ID NO: 5 - DNA, 408 bp, Mus musculus
atggattttgggctgattttttttattgttgctcttttaaaaggggtccagtgtgaggtgaagcttctcgagtctggaggtggcctggtgcagcctggaggatccctgaaagtctcctgtgcagcctcaggattcgattttagtagatactggatgagttgggtccggcaggctccagggaaagggctagaatggattggagaaattaatccagatagcagtaagataaactatacgccatctctaaaggataaattcatcatctccagagacaacgccaaaaatacgctgtacctgcaaatggacaaagtgacatctgaggacacagccctttattgctgtgcaagaccggagggctactggtacttggatgtctggggcgcagggaccacggtcaccgtctcctca

SEQ ID NO: 6 - protein, 136 aa, Mus musculus
MDFGLIFFIVALLKGVQCEVKLLESGGGLVQPGGSLKVSCAASGFDFSRYWMSWVRQAPGKGLEWIGEINPDSSKINYTPSLKDKFIISRDNAKNTLYLQMDKVTSEDTALYCCARPEGYWYLDVWGAGTTVTVSS

SEQ ID NO: 7 - DNA, 393 bp, Mus musculus
atgggcatcaaaatggagtcacagattcaggtctttgtattcgtgtttctctggttgtctggtgttgacggagacattgtgatgacccagtctcacaaattcatgtccacatcagtaggagacagggtcaacatcacctgcaaggccagtcagggtgtgagaactgctatagcctggtatcaacagaaaccaggacaatctcctaaactactgttttactcggcatcctaccggtacactggagtccctgatcgcttcactggcagtggatctgggacggctttcactttcaccatcagcagtgtgcaggctgaagacctggcagtttatttctgtcagcaacattatagtactccgtacacgttcggaggcgggaccaagctggaaataaaa

SEQ ID NO: 8 - protein, 131 aa, Mus musculus
MGIKMESQIQVFVFVFLWLSGVDGDIVMTQSHKFMSTSVGDRVNITCKASQGVRTAIAWYQQKPGQSPKLLFYSASYRYTGVPDRFTGSGSGTAFTFTISSVQAEDLAVYFCQQHYSTPYTFGGGTKLEIK

SEQ ID NO: 9 - protein, 6 aa, Mus musculus
SDYAWN

SEQ ID NO: 10 - protein, 16 aa, Mus musculus
YISYSGSTNYNPSLKS

SEQ ID NO: 11 - protein, 4 aa, Mus musculus
DGGY

SEQ ID NO: 12 - protein, 16 aa, Mus musculus
KSSQSLLYSSNQKNYL

SEQ ID NO: 13 - protein, 7 aa, Mus musculus
WASTRES

SEQ ID NO: 14 - protein, 9 aa, Mus musculus
QQYYSYPLT

SEQ ID NO: 15 - protein, 5 aa, Mus musculus
RYWMS

SEQ ID NO: 16 - protein, 17 aa, Mus musculus
EINPDSSKINYTPSLKD

SEQ ID NO: 17 - protein, 9 aa, Mus musculus
PEGYWYLDV

SEQ ID NO: 18 - protein, 11 aa, Mus musculus
KASQGVRTAIA

SEQ ID NO: 19 - protein, 7 aa, Mus musculus
SASYRYT

SEQ ID NO: 20 - protein, 9 aa, Mus musculus
QQHYSTPYT

SEQ ID NO: 21 - protein, 113 aa, Mus musculus
DVQLQESGPGLVKPSQSLSLTCTVTGYSITSDYAWNWIRQFPGNKLEWMGYISYSGSTNYNPSLKSRISITRDTSKNQFFLQLNSVTTEDTATYYCTRDGGYWGQGTSVTVSS

SEQ ID NO: 22 - protein, 113 aa, Mus musculus
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLELK

SEQ ID NO: 23 - protein, 118 aa, Mus musculus
EVKLLESGGGLVQPGGSLKVSCAASGFDFSRYWMSWVRQAPGKGLEWIGEINPDSSKINYTPSLKDKFIISRDNAKNTLYLQMDKVTSEDTALYCCARPEGYWYLDVWGAGTTVTVSS

SEQ ID NO: 24 - protein, 107 aa, Mus musculus
DIVMTQSHKFMSTSVGDRVNITCKASQGVRTAIAWYQQKPGQSPKLLFYSASYRYTGVPDRFTGSGSGTAFTFTISSVQAEDLAVYFCQQHYSTPYTFGGGTKLEIK

(19) United States
(12) Patent Application Publication
(10) Pub. No.: US 2018/0298094 A1
(43) Pub. Date: Oct. 18, 2018

(54) MONOCLONAL ANTIBODIES TARGETED TO HUMAN TAXILIN ALPHA AND METHODS FOR USE OF SAME

(71) Applicant: KSL BIOMEDICAL LLC, Clarence, NY (US)

(72) Inventors: Long SHEN, Williamsville, NY (US); Lakshmanan SURESH, Williamsville, NY (US)

(73) Assignee: KSL BIOMEDICAL LLC

(21) Appl. No.: 15/952,768

(22) Filed: Apr. 13, 2018

Related U.S. Application Data
(60) Provisional application No. 62/486,126, filed on Apr. 17, 2017.

(51) Int. Cl. C07K 16/24; G01N 33/68

(52) U.S. Cl. CPC C07K 16/244; G01N 33/6869; G01N 2800/101; C07K 2317/56; G01N 2333/544; C07K 2317/565

(57) ABSTRACT

A method for diagnosis or aiding in the diagnosis of Sjögren's Syndrome (SS). The diagnosis method requires testing a sample obtained or derived from a subject for an amount of Taxilin alpha, wherein determining an amount of Taxilin alpha that is more than a reference comprises a diagnosis or aids in a diagnosis that the individual has SS, and wherein determining an amount of Taxilin alpha that is the same as or less than a reference indicates the individual does not have SS. A mAb or Taxilin alpha-binding fragment thereof (“Taxilin alpha”) having an antibody heavy chain; an antibody light chain, an antibody comprising a heavy chain and/or an antibody. A kit of the same monoclonal antibody or Taxilin alpha-binding fragment thereof. A method of making the Taxilin alpha. The making method requires separating the Taxilin alpha from a cell culture that expresses the Taxilin alpha.

### FIELD OF THE INVENTION

The present disclosure relates generally to monoclonal antibodies (mAbs) and antigen binding fragments of them that bind with specificity to human Taxilin alpha. The mAbs are for use with diagnostic and therapeutic implementations.

### BACKGROUND OF THE INVENTION

### 1. Sjögren's Syndrome:

Sjögren's syndrome (SS) is a chronic, systemic inflammatory disorder that mainly affects the exocrine glands with a typical focal lymphocytic infiltration potentially leading to dry mouth (xerostomia) and dry eyes (xerophthalmia). While sicca symptoms are the hallmarks of the syndrome, during the disease development, patients might experience various systemic clinical manifestations (i.e., fatigue, arthritis, skin vasculitis, hematological disorders, lung interstitial diseases, kidney failure, peripheral and central neuropathies and gastrointestinal tract disorders). As a result, SS was considered a heterogeneous autoimmune disease possessing both organ-specific and systemic features and encompassing a wide spectrum of clinical/serological abnormalities and scattered complications.

Sjögren's syndrome is one of the most common autoimmune disease in adults affecting up to 3.2 M cases in the United States. Previous studies demonstrated that about 1 in 10 patients with clinically significant dry eye have underlying SS. However, SS is greatly under recognized in clinical practice, mostly due to diverse symptomatic expressions making the initial diagnosis difficult. It is estimated that the disease remains undiagnosed in more than half of affected adults. While currently there is no cure for SS, results from recent clinical studies with rituximab for patients with primary SS and severe systemic complications were promising. The studies showed rituximab improves salivary gland function, diminishes fatigue, and reduces the number of extra-glandular manifestations, especially when the treatment was initiated early in the disease course. This underlines the importance of early diagnosis to identify SS patients before irreversible damage to the affected organs and tissues occur.

### 2. Taxilin

Taxilin is commonly referred to as IL-14. Taxilin is a cytokine that was originally identified and cloned from a Burkitt lymphoma cell line and shown to enhance B cell proliferation, especially of germinal center B cells and surface Ig (ₛIg)D^{low} human tonsillar B cells, which includes B1 cells and activated B2 cells. The NCBI has designated the Taxilin gene Tx1n. The Taxilin alpha transcript is produced from the plus strand of the Taxilin gene using exons 3-10.

Taxilin alpha induces Sjögren's disease by converting low affinity autoreactivity into high affinity memory B cell responses. The Taxilin alpha transgenic mouse spontaneously develops SS with many of the features seen in patients in the same relative time frame.

### SUMMARY OF THE INVENTION

A mAb or Taxilin alpha-binding fragment thereof having an antibody heavy chain; an antibody light chain, an antibody comprising a heavy chain and/or an antibody. A kit of the same monoclonal antibody or Taxilin alpha-binding fragment thereof. The antibody heavy chain having one, two or three of the following mAb-1 CDRs: CDR1: SDYAWN; CRR2: YISYSGSTNYNPSLKS; and CDR3: DGGY. The antibody light chain having one, two or three of the following mAb-1 CDRs: CDR1: KSSQSLLYSSNQKNYL; CRR2: WASTRES; and CDR3: QQYYSYPLT. The antibody having a heavy chain having one, two or three of the following mAb-2 CDRs: CDR1: RYWMS; CRR2: EINPDSSKINYTPSLKD; and CDR3: PEGYWYLDV. The antibody comprising a light chain having one, two or three of the following mAb-2 CDRs: CDR1: KASQGVRTAIA; CRR2: SASYRYT; and CDR3: QQHYSTPYT.

A method of making the above-identified monoclonal antibody or Taxilin alpha-binding fragment thereof. The making method requires separating the monoclonal antibody or Taxilin alpha-binding fragment thereof from a cell culture that expresses the monoclonal antibody or the Taxilin alpha-binding fragment thereof.

A method for diagnosis or aiding in the diagnosis of Sjögren's Syndrome (SS). The diagnosis method requires testing a sample obtained or derived from a subject for an amount of Taxilin alpha, wherein determining an amount of Taxilin alpha that is more than a reference comprises a diagnosis or aids in a diagnosis that the individual has SS, and wherein determining an amount of Taxilin alpha that is the same as or less than a reference indicates the individual does not have SS.

### BRIEF DESCRIPTION OF THE FIGURES

FIG. 1 is a photograph for evaluating serum level of Taxilin alpha protein in SS by western blots.

FIGS. 2A and 2B illustrate by graph the evaluation of serum level of Taxilin alpha and BAFF in different disease groups;

FIGS. 3A, 3B, and 3C illustrate through graphs comparison of serum level of Taxilin alpha in different disease groups by age; and

FIGS. 4A and 4B illustrate through graphs in pSS patients, the serum level of Taxilin alpha decrease whereas BAFF level increase as age increases.

### DEFINITIONS

The term “biological sample” refers to a body sample from any animal, but preferably is from a mammal, more preferably from a human. In certain embodiments, said biological sample is from a patient with signs of an autoimmune disorder. Such samples include biological fluids such as serum, plasma, vitreous fluid, lymph fluid, synovial fluid, amniotic fluid, whole blood, urine, saliva, sputum, tears, perspiration, mucus, tumor lysates and tissue culture medium, as well as tissue extracts such a homogenized tissue, tumor tissue and cellular extracts. In certain embodiments, the sample is a body sample from any animal; in one embodiment it is from a mammal; and in another embodiment it is from a human subject. In one embodiment, such biological sample is from clinical patients.

The term “detecting” is used in the broadest sense to include both qualitative and quantitative measurement of a target molecule. In one aspect, the detecting method as described herein is used to identify the mere presence of Taxilin.

The term “detectable antibody” refers to an antibody that is capable of being detected either directly through a label amplified by detection means, or indirectly through, e.g., another antibody that is labeled. For direct labeling, the antibody is typically conjugated to a moiety that is detectable by some means. In one embodiment, the detectable antibody is a monoclonal antibody.

The term “detection means” refers to a moiety or technique used to detect the presence of the detectable antibody in the assay herein and depends on the type of label that is used and the format of the immunoassay. For example, ELISA (defined below) assays include detection agents that amplify the immobilized label such as label captured onto a microtiter plate, such as a colorimetric detection agent.

The term “capture reagent” refers to a reagent capable of binding and capturing a target molecule in a sample such that under suitable conditions, the capture reagent-target molecule complex can be separated from the rest of the sample. Typically, the capture reagent is immobilized or immobilizable. In a sandwich immunoassay, the capture reagent is preferably an antibody or a mixture of different antibodies against a target antigen.

The term “antibody” herein is used in the broadest sense and specifically covers intact monoclonal antibodies, polyclonal antibodies, multispecific antibodies (e.g., bispecific antibodies) formed from at least two intact antibodies, and antibody fragments so long as they exhibit the desired biological activity.

The term “antibody fragments” comprises a portion of an intact antibody, preferably comprising the antigen-binding or variable region thereof. Examples of antibody fragments include Fa, Fab′, F(ab′)2, and Fv fragments (defined in greater detail below); diabodies; linear antibodies; single-chain antibody molecules; and multispecific antibodies formed from antibody fragments. For the purposes herein, an “intact antibody” is one comprising heavy- and light-chain variable domains as well as an Fc region (defined in greater detail below).

The term “native antibodies” are usually heterotetrameric glycoproteins of about 150,000 daltons, composed of two identical light (L) chains and identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. In contrast to conventional (polyclonal) antibody preparations that typically include different antibodies directed against different determinants (epitopes), each monoclonal antibody is directed against a single determinant on the antigen. In addition to its specificity, the monoclonal antibodies are advantageous in that they are synthesized by the hybridoma culture, uncontaminated by other immunoglobulins. The modified “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primatized” antibodies comprising variable-domain antigen-binding sequences derived from a non-human primate and human constant-region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Humanized antibodies may comprise residues that are not found in the recipient antibody or in the donor antibody. These modifications are made further to refine antibody performance. In general, the humanized antibody will comprise residues that are not found in the recipient antibody or in the donor antibody. These modifications are made further to refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see, Jones et al., Nature, 321: 522-525 (1986); Riechmann et al., Nature, 332:323-329 (1988); and Presta, Curr. Op. Struct. Biol., 2:593-596 (1992). In one embodiment, a humanized monoclonal antibody to salivary gland protein 1 (SP-1) or fragments thereof is provided and used the methods provided herein. In another embodiment, a humanized monoclonal antibody to parotid secretory protein (PSP) or fragments thereof is provided and used in the methods provided herein. In yet another embodiment, a humanized monoclonal antibody to carbonic anhydrase 6 (CA6) or fragments thereof is provided and used in the methods provided herein.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions in both the light-chain and the heavy-chain variable domains. The more highly conserved portions of variable domains are called the framework regions (FRs). The variable domains of native heavy an light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment that contains a complete antigen-recognition and antigen-binding site. This region consists of a dimer of one heavy-chain and one light-chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy-chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ϵ, γ and μ, respectively. The subunit structures and three-dimensional configurations of different classes of immunoglobulins are well known.

“Single-chain Fv” or “ScFv” antibody fragments comprise the VH and VL domains of antibody, wherein these domains are present in a single polypeptide chain. Preferably, the Fv and VH and VL domains that enables the scFv to form the desired structure for antigen binding. For a review of scFv, see, Pluckthun in The Pharmacology of Monoclonal Antibodies, vol 113, Rosenburg and Moore, eds., Springer-Verlag, New York, pp. 269-315 (1994).

The term “hypervariable region” when used herein refers to the amino acid residues of an antibody that are responsible for antigen binding. The hypervariable region comprises amino acid residues from a “complementary-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light-chain variable domain and 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 196: 901-917 (1987)). “Framework” or “FR” residues are those variable domain residues other than the hypervariable region residues as herein defined.

“Mammal” for purposes of treatment refers to any animal classified as a mammal, including humans, domestic, and farm animals, and zoo, sports, or pet animals, such as dogs, horses, cats, sheep, pigs, cows, etc. Preferably, the mammal is human.

The term “affinity purified” refers to purifying a substance by eluting it through an affinity chromatography column.

The term “ELISA” as used herein refers to enzyme-linked immunosorbent assays (ELISAs) for various antigens, including biomarkers for SS, which include those based on colorimetry, chemiluminescence, and fluorometry. ELISAs have been successfully applied in the determination of low amounts of drugs and other antigenic components in plasma and urine samples, involve no extraction steps, and are simple to carry out.

### Part 1. Monoclonal Antibodies for Human Taxilin Alpha Protein

Murine monoclonal antibodies against human Taxilin alpha protein were generated through contract service with PTGLAB. The recombinant Taxilin alpha protein used to raise the Abs were made by a clone with construct containing a 933 bp PCR product (Genebank accession number: BC103823, cloned cDNA region: 670-1602nt) inserted into PET30a vector.

Two hybridoma lines were selected based on test result from Western blot and ELISA, the CDR sequence for the first hybridomas was listed as below:

Heavy chain: DNA sequence (393 bp)
Leader sequence-FR1-CDR1-FR2-CDR2-FR3-CDR3-FR4
ATGAGAGTGCTGATTCTTTTGTGGCTGTTCACAGCCTTTCCTGGTA
TCCTGTCTGATGTGCAGCTTCAGGAGTCGGGACCTGGCCTGGTGAA
ACCTTCTCAGTCTCTGTCCCTCACCTGCACTGTCACTGGCTACTCA
ATCACCAGTGATTATGCCTGGAACTGGATCCGGCAGTTTCCAGGAA
ACAAACTGGAGTGGATGGGCTACATAAGCTACAGTGGTAGCACTAA
CTACAACCCATCTCTCAAAAGTCGAATCTCTATCACTCGAGACACA
TCCAAGAACCAGTTCTTCCTGCAGTTGAATTCTGTGACTACTGAGG
ACACAGCCACATATTACTGTACAAGAGATGGGGGTTACTGGGGTCA
AGGAACCTCAGTCACCGTCTCCTCA
Heavy chain: Amino acids sequence (131 aa)
Leader sequence-FR1-CDR1-FR2-CDR2-FR3-CDR3-FR4
MRVLILLWLFTAFPGILSDVQLQESGPGLVKPSQSLSLTCTVTGYS
ITSDYAWNWIRQFPGNKLEWMGYISYSGSTNYNPSLKSRISITRDT
SKNQFFLQLNSVTTEDTATYYCTRDGGYWGQGTSVTVSS
Light chain: DNA sequence (399 bp)
Leader sequence-FR1-CDR1-FR2-CDR2-FR3-CDR3-FR4
ATGGATTCACAGGCCCAGGTTCTTATGTTACTGCTGCTATGGGTAT
CTGGTACCTGTGGGGACATTGTGATGTCACAGTCTCCATCCTCCCT
AGCTGTGTCAGTTGGAGAGAAGGTTACTATGAGCTGCAAGTCCAGT
CAGAGCCTTTTATATAGTAGCAATCAAAAGAACTACTTGGCCTGGT
ACCAGCAGAAACCAGGGCAGTCTCCTAAACTGCTGATTTACTGGGC
ATCCACTAGGGAATCTGGGGTCCCTGATCGCTTCACAGGCAGTGGA
TCTGGGACAGATTTCACTCTCACCATCAGCAGTGTGAAGGCTGAAG
ACCTGGCAGTTTATTACTGTCAGCAATATTATAGCTATCCTCTCAC
GTTCGGTGCTGGGACCAAGCTGGAGCTGAAA
Light chain: Amino acids sequence (133 aa)
Leader sequence-FR1-CDR1-FR2-CDR2-FR3-CDR3-FR4
MDSQAQVLMLLLLWVSGTCGDIVMSQSPSSLAVSVGEKVTMSCKSS
QSLLYSSNQKNYLAWYQQKPGQSPKLLIYWASTRESGVPDRFTGSG
SGTDFTLTISSVKAEDLAVYYCQQYYSYPLTFGAGTKLELK

The CDR sequence for the second hybridomas was listed as below:

Heavy chain: DNA sequence (408 bp)
Leader sequence-FR1-CDR1-FR2-CDR2-FR3-CDR3-FR4
ATGGATTTTGGGCTGATTTTTTTTATTGTTGCTCTTTTAAAAGGGG
TCCAGTGTGAGGTGAAGCTTCTCGAGTCTGGAGGTGGCCTGGTGCA
GCCTGGAGGATCCCTGAAAGTCTCCTGTGCAGCCTCAGGATTCGAT
TTTAGTAGATACTGGATGAGTTGGGTCCGGCAGGCTCCAGGGAAAG
GGCTAGAATGGATTGGAGAAATTAATCCAGATAGCAGTAAGATAAA
CTATACGCCATCTCTAAAGGATAAATTCATCATCTCCAGAGACAAC
GCCAAAAATACGCTGTACCTGCAAATGGACAAAGTGACATCTGAGG
ACACAGCCCTTTATTGCTGTGCAAGACCGGAGGGCTACTGGTACTT
GGATGTCTGGGGCGCAGGGACCACGGTCACCGTCTCCTCA
Heavy chain: Amino acids sequence (136 aa)
Leader sequence-FR1-CDR1-FR2-CDR2-FR3-CDR3-FR4
MDFGLIFFIVALLKGVQCEVKLLESGGGLVQPGGSLKVSCAASGFD
FSRYWMSWVRQAPGKGLEWIGEINPDSSKINYTPSLKDKFIISRDN
AKNTLYLQMDKVTSEDTALYCCARPEGYWYLDVWGAGTTVTVSS
Light chain: DNA sequence (393 bp)
Leader sequence-FR1-CDR1-FR2-CDR2-FR3-CDR3-FR4
ATGGGCATCAAAATGGAGTCACAGATTCAGGTCTTTGTATTCGTGT
TTCTCTGGTTGTCTGGTGTTGACGGAGACATTGTGATGACCCAGTC
TCACAAATTCATGTCCACATCAGTAGGAGACAGGGTCAACATCACC
TGCAAGGCCAGTCAGGGTGTGAGAACTGCTATAGCCTGGTATCAAC
AGAAACCAGGACAATCTCCTAAACTACTGTTTTACTCGGCATCCTA
CCGGTACACTGGAGTCCCTGATCGCTTCACTGGCAGTGGATCTGGG
ACGGCTTTCACTTTCACCATCAGCAGTGTGCAGGCTGAAGACCTGG
CAGTTTATTTCTGTCAGCAACATTATAGTACTCCGTACACGTTCGG
AGGCGGGACCAAGCTGGAAATAAAA
Light chain: Amino acids sequence (131 aa)
Leader sequence-FR1-CDR1-FR2-CDR2-FR3-CDR3-FR4
MGIKMESQIQVFVFVFLWLSGVDGDIVMTQSHKFMSTSVGDRVNIT
CKASQGVRTAIAWYQQKPGQSPKLLFYSASYRYTGVPDRFTGSGSG
TAFTFTISSVQAEDLAVYFCQQHYSTPYTFGGGTKLEIK

### Part 2. Evaluating Serum Level of Taxilin Alpha Protein as Diagnostic Biomarker for Sjögren's Syndrome

Serum samples were collected from 10 confirmed SS patients and 6 Healthy controls (“NC”), western blots assay was used to evaluate the expression level of Taxilin alpha protein. As shown in FIG. 1—Evaluating Serum Level of Taxilin alpha Protein in SS by Western Blots—and confirmed in the data expressed at Table 1, all 10 SS patients has elevated level of Taxilin alpha protein. Further analysis was done by Quantity One® 1-D Analysis Software (Bio-Rad) to measure the intensity of the band in WB and define the value of mean density Table 1. The sensitivity and specificity of this assay were outlined in table 2.

TABLE 1
Mean Intensity value of Taxilin alpha WB result

| Lane | Sample ID | Mean Density value |
|---|---|---|
| 1 | NC1 | 126.82 |
| 2 | NC2 | 78.37 |
| 3 | NC3 | 79.95 |
| 4 | NC4 | 59.95 |
| 5 | NC5 | 65.63 |
| 6 | NC6 | 99.75 |
| 7 | SS1 | 172.52 |
| 8 | SS2 | 196.20 |
| 9 | SS3 | 133.55 |
| 10 | SS4 | 174.81 |
| 11 | SS5 | 123.95 |
| 12 | SS6 | 160.14 |
| 13 | SS7 | 168.82 |
| 14 | SS8 | 164.35 |
| 15 | SS9 | 203.30 |
| 16 | SS10 | 237.30 |

TABLE 2
Sensitivity and Specificity of the Taxilin alpha WB assay

| Cutoff define | Cutoff value | Sensitivity | Specificity |
|---|---|---|---|
| Average + 1SD | 110 | 100% | 83% |
| Average + 2SD | 134 | 90% | 100% |

### Supporting Clinical Study Data

### Taxilin as a Putative Biomarker for Stratification of Dry Eyes in Primary Sjögren's Syndrome

The pathogenesis of primary Sjögren's syndrome (pSS) is associated with abnormal B cell activation, resulting in production of excessive autoantibodies and disorder to the cytokine network. Taxilin is a cytokine that was shown to enhance B cell proliferation, especially of germinal center B cells. Transgenic mice overexpressing human Taxilin alpha can develop many clinical features of pSS in the same relative time frame as seen in patients. While upregulation of Taxilin alpha gene expression has been shown in the peripheral blood leukocytes of pSS patients, till now the measurement of Taxilin alpha serum levels has not been possible due to lack of validated assay. Taxilin alpha has been determined to be a biomarker and its correlation to B cell activating factor (BA F), a well-established cytokine in pSS through upregulating innate immune activation and chronic autoimmune B cell activation, in a cohort of patient with non-SS dry eye (NSDE), pSS, diseases and healthy controls (HC).

### Methods:

Total of 181 fresh serum samples were collected and stored in −80 degrees freezer. Among them, 65 were pSS patients (age 53.15±14.08 years) who meet the 2012 ACR Classification Criteria for Sjögren's Syndrome, 20 were dry eye patients excluding SS (age 44.85±11.39 years, NSDE), 50 were Rheumatoid Arthritis patients (age 54.95±15.35 years, RA) and 46 were healthy controls (age 43.49±14.57 years, HC). See, Table 1. Serum level of Taxilin alpha was evaluated by quantitative Western Blots assay and BAFF level was evaluated by ELISA assay (R&D System). All clinical and laboratory data were reviewed following protocol approved by Peking University People's Hospital IRB committee. Statistical analysis was done by software Prism 6.0 with unpaired t tests.

TABLE 1
Basic Clinical Characteristics of Study Groups

| | Total Case | Sex(male/ | |
|---|---|---|---|
| Group | Number | female) | Age |
| Healthy Control (HC) | 45 | 18/27 | 43.27 ± 14.78 |
| Sjögren's Syndrome (SS) | 65 | 2/63 | 53.15 ± 14.08 |
| Rheumatoid Arthritis (RA) | 50 | 17/33 | 53.68 ± 15.26 |
| Non-SS dry eye (NSDE) | 20 | 0/20 | 44.85 ± 11.39 |

### Results:

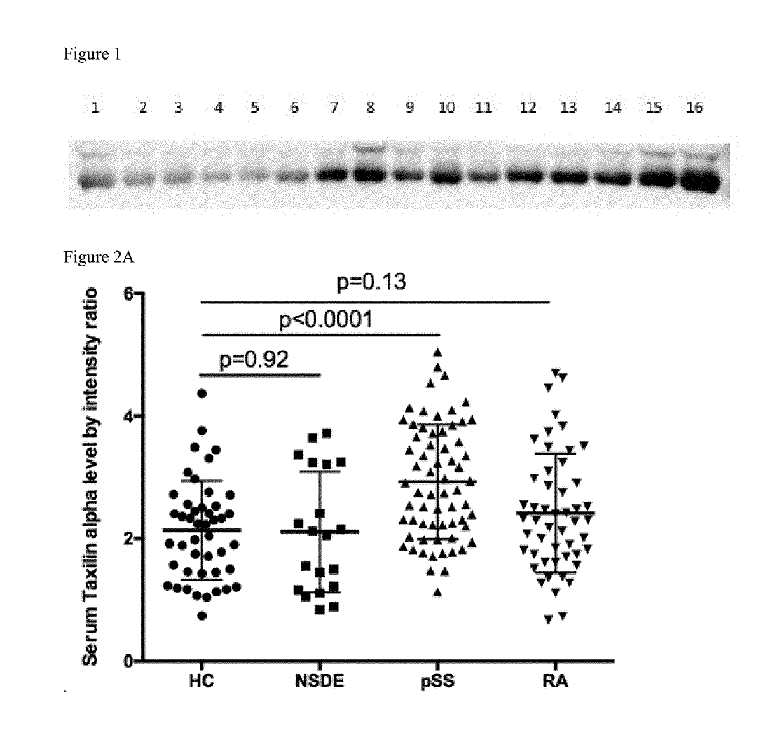

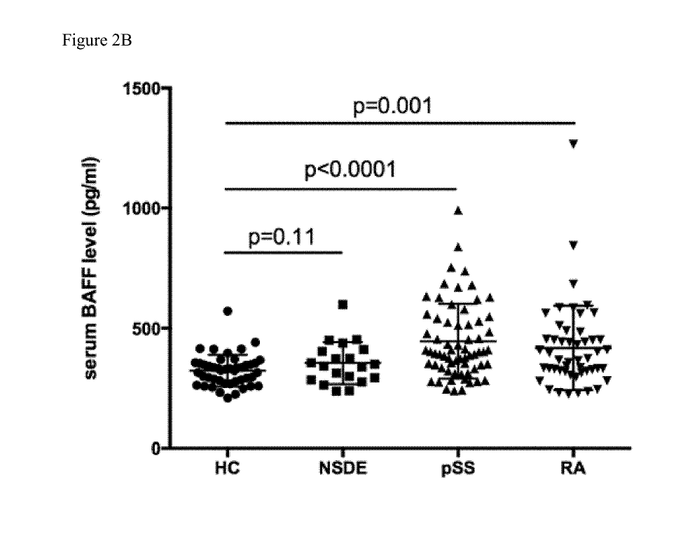

As illustrated at FIGS. 2A and 2B, after normalized with internal control, the relative intensity ratio for serum Taxilin alpha level in HC group was 2.13±0.81, NSDE group was 2.11±0.98 (p=0.99), pSS group was 2.92±0.93 (p<0.0001) and RA group was 2.47±0.95 (p=0.15). Serum BAFF level (pg/ml) in HC group was 323.56±65.85, DE group was 355.21±87.86 (p=0.22), pSS group was 455.94±155.16 (p<0.0001) and RA group was 448.38±220.07 (p=0.0002).

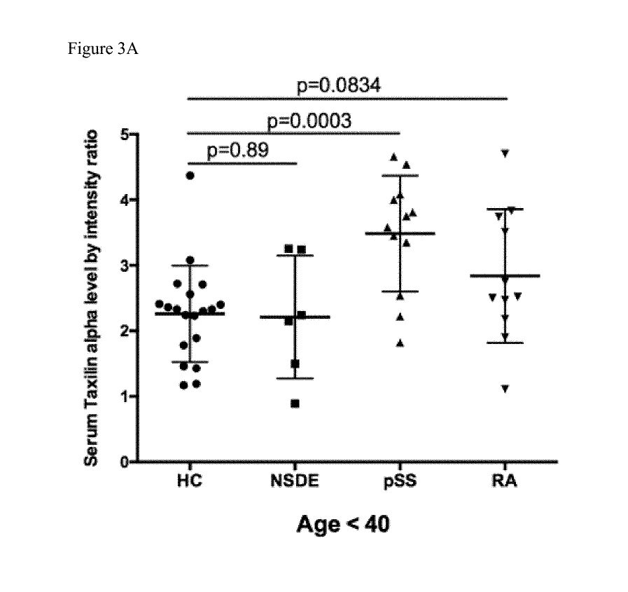

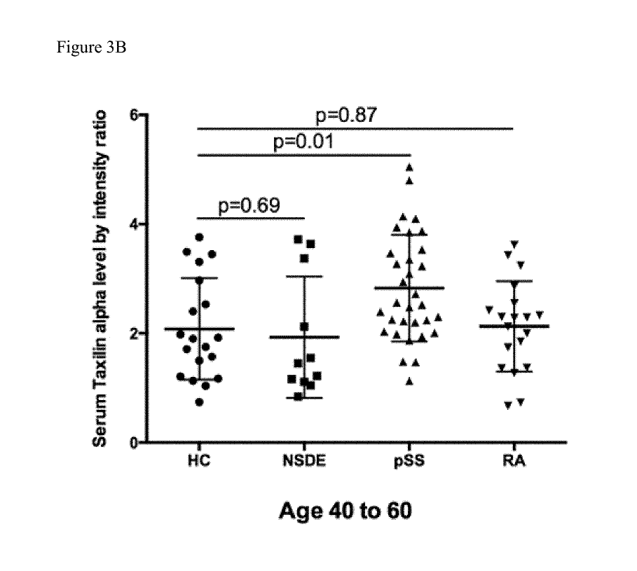

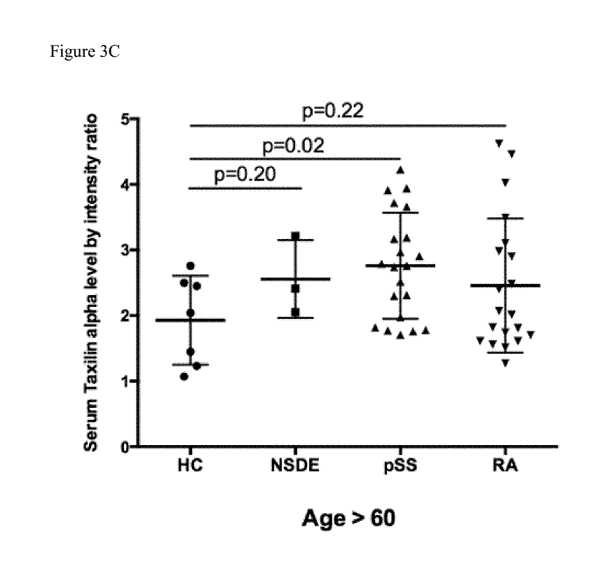

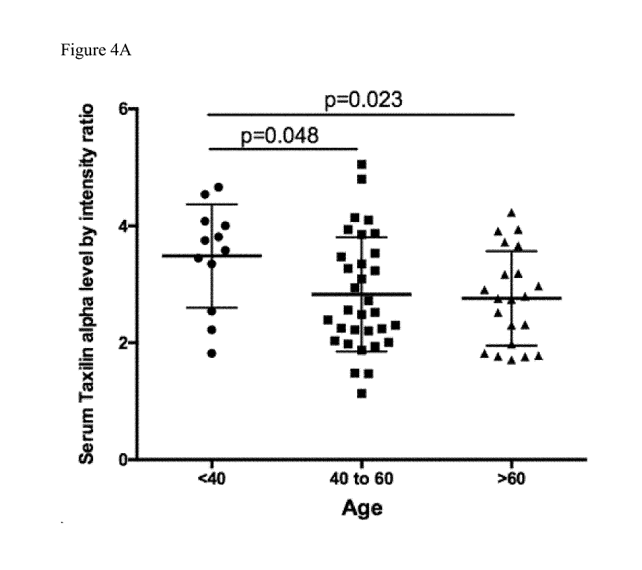

As illustrated at FIGS. 3A, 3B, and 3C, for age <40 years, the serum level of Taxilin alpha in HC group was 2.26±0.73, NSDE group was 2.21±0.93 (p=0.89), pSS group was 3.48±0.88 (p=0.0003) and RA group was 3.28±0.87 (p=0.08). For age 40 to 60 years, the serum level of HC group was 2.08±0.93, NSDE group was 1.93±1.11 (p=0.69), pSS group was 2.83±0.98 (p=0.01) and RA group was 2.23±0.76 (p=0.87). For age >60 years, the serum level of HC group was 1.93±0.68, NSDE group was 2.56±0.59 (p=0.21), pSS group was 2.76±0.81 (p=0.02) and RA group was 2.49±1.04 (p=0.12).

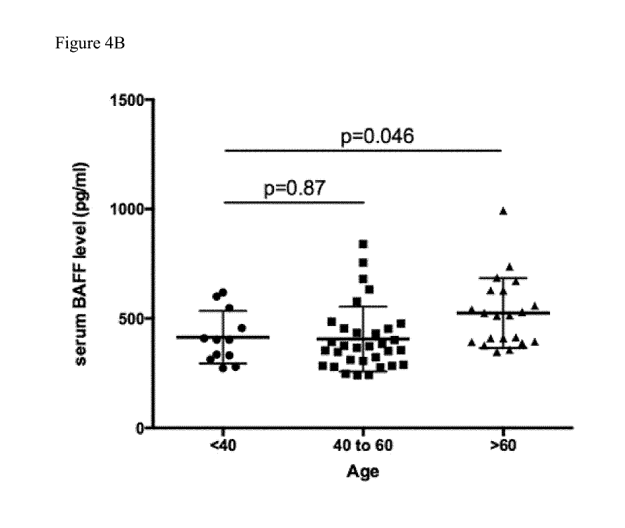

As illustrated at FIGS. 4A and 4B, in pSS patients, the serum level of Taxilin alpha decrease as age increase (<40 years, 3.48±0.88, 40-60 years, 2.83±0.98, (p=0.048) and >60 years, 2.76±0.81, (p=0.023)). Whereas the serum level of BAFF (pg/ml) increase as age increases (<40 years, 414.22±119.94, 40-60 years, 406.22±148.29, (p=0.87), >60 years, 524.57±159.46, (p=0.008)).

In conclusion, elevation of serum Taxilin alpha level is a key cytokine biomarker for the stratification of SS vs NSDE.

Taxilin alpha and BAFF work in different fashions to maintain the abnormal B cell activation as seen is pSS patients.

The above-identified method for diagnosis or aiding in the diagnosis of Sjögren's Syndrome (SS) can also be performed in the following conventional assays to obtain the same results: western blot, immunofluorescense assay, enzyme immuno-assay, chemiluminiscence assay, flow cytometry assay, line immuno-assay, and immunohistochemistry assay.

While the invention has been described through specific embodiments, routine modifications will be apparent to those skilled in the art and such modifications are intended to be within the scope of the present invention.

1. A mAb or Taxilin alpha-binding fragment thereof comprising:
an antibody heavy chain comprising one, two or three of the following mAb-1 CDRs:
CDR1:
SDYAWN
CDR2:
YISYSGSTNYNPSLKS
CDR3:
DGGY
and/or an antibody light chain comprising one, two or three of the following mAb-1 CDRs:
CDR1:
KSSQSLLYSSNQKNYL
CDR2:
WASTRES
CDR3:
QQYYSYPLT
and/or an antibody comprising a heavy chain having one, two or three of the following mAb-2 CDRs:
CDR1:
RYWMS
CDR2:
EINPDSSKINYTPSLKD
CDR3:
PEGYWYLDV
and/or an antibody comprising a light chain having one, two or three of the following mAb-2 CDRs:
CDR1:
KASQGVRTAIA
CDR2:
SASYRYT
CDR3:
QQHYSTPYT.

2. The mAb or Taxilin alpha-binding fragment of claim 1 comprising a heavy chain comprising the three mAb-1 CDRs:
CDR1:
SDYAWN
CDR2:
YISYSGSTNYNPSLKS
CDR3:
DGGY
and/or an antibody light chain comprising the three CDRs:
CDR1:
KSSQSLLYSSNQKNYL
CDR2:
WASTRES
CDR3:
QQYYSYPLT.

3. The mAb or Taxilin alpha-binding fragment of claim 1 comprising a heavy chain comprising the three mAb-2 CDRs:
CDR1:
RYWMS
CDR2:
EINPDSSKINYTPSLKD
CDR3:
PEGYWYLDV
and/or an antibody light chain comprising the three mAb-2 CDRs:
CDR1:
KASQGVRTAIA
CDR2:
SASYRYT
CDR3:
QQHYSTPYT.

4. A mAb or Taxilin alpha-binding fragment thereof comprising a heavy chain variable region comprising the sequence:
DVQLQESGPGLVKPSQSLSLTCTVTGYSITSDYAWNWIRQFPGNKL
EWMGYISYSGSTNYNPSLKSRISITRDTSKNQFFLQLNSVTTEDTA
TYYCTRDGGYWGQGTSVTVSS
and/or a light chain variable region comprising the sequence:
DIVMSQSPSSLAVSVGEKVTMSCKSSQSLLYSSNQKNYLAWYQQKP
GQSPKLLIYWASTRESGVPDRFTGSGSGTDFTLTISSVKAEDLAVY
YCQQYYSYPLTFGAGTKLELK.

5. A mAb or Taxilin alpha-binding fragment thereof comprising a heavy chain variable region comprising the sequence:
EVKLLESGGGLVQPGGSLKVSCAASGFDFSRYWMSWVRQAPGKGLE
WIGEINPDSSKINYTPSLKDKFIISRDNAKNTLYLQMDKVTSEDTA
LYCCARPEGYWYLDVWGAGTTVTVSS
and/or a light chain variable region comprising the sequence:
DIVMTQSHKFMSTSVGDRVNITCKASQGVRTAIAWYQQKPGQSPKL
LFYSASYRYTGVPDRFTGSGSGTAFTFTISSVQAEDLAVYFCQQHY
STPYTFGGGTKLEIK.

6. A complex comprising a mAb or Taxilin alpha-binding fragment thereof of claim 1 non-covalently bound to Taxilin alpha.

7. An expression vector encoding a mAb or Taxilin alpha-binding fragment thereof of claim 1.

8. A cell culture comprising the expression vector of claim 7.

9. A hybridoma that produces a monoclonal antibody of claim 1.

10. A kit comprising a monoclonal antibody or Taxilin alpha-binding fragment thereof of claim 1.

11. A method of making a monoclonal antibody or Taxilin alpha-binding fragment thereof of claim 1 comprising separating the monoclonal antibody or Taxilin alpha-binding fragment thereof from a cell culture that expresses the monoclonal antibody or the Taxilin alpha-binding fragment thereof.

12. A method for diagnosis or aiding in the diagnosis of Sjögren's Syndrome (SS) comprising testing a sample obtained or derived from a subject for an amount of Taxilin alpha, wherein determining an amount of Taxilin alpha that is more than a reference comprises a diagnosis or aids in a diagnosis that the individual has SS, and wherein determining an amount of Taxilin alpha that is the same as or less than a reference indicates the individual does not have SS.

13. The method of claim 12 wherein the testing comprises immunologically determining the amount of the Taxilin alpha.

14. The method of claim 12, wherein the immunologically determining comprises detecting a complex of the Taxilin alpha and a mAb or an Taxilin alpha binding fragment of the mAb.

15. The method of claim 12, wherein the testing comprises an enzyme-linked immunosorbent assay (ELISA) assay, wherein the ELISA assay is performed using a mAb or an Taxilin alpha binding fragment of the mAb.

16. The method of claim 12 wherein the method performed in an assay selected from the group consisting of western blot, immunofluorescense assay, enzyme immuno-assay, chemiluminiscence assay, flow cytometry assay, line immuno-assay, and immunohistochemistry assay.
